(19) United States
(12) Patent Application Publication
(10) Pub. No.: US 2025/0360314 A1
(43) Pub. Date: Nov. 27, 2025

(54) ADAPTIVE SYNCHRONIZATION OF ORIENTATION-SPECIFIC CORTICAL OSCILLATIONS FOR THERAPEUTIC NEUROMODULATION

(71) Applicant: Brain Electrophysiology Laboratory Company, LLC, Eugene, OR (US)

(72) Inventors: Don M. Tucker, Eugene, OR (US); Phan Luu, Eugene, OR (US); Roman Shusterman, Lake Oswego, OR (US)

(21) Appl. No.: 19/271,007

(22) Filed: Jul. 16, 2025

Related U.S. Application Data
(62) Division of application No. 19/181,619, filed on Apr. 17, 2025.

(51) Int. Cl. A61N 1/36; A61B 5/00; A61B 5/372; G16H 20/40; G16H 50/30

(52) U.S. Cl. CPC A61N 1/36025; A61B 5/372; A61B 5/4094; A61B 5/4836; A61N 1/36031; A61N 1/36034; G16H 20/40; G16H 50/30

(57) ABSTRACT

A method for modulating the electrical synchronization of the cerebral cortex within safe limits by computing the orientation of the applied currents with respect to the orientation of the cortical surface and thus cortical columns. Ongoing electroencephalographic monitoring, including the synchronization of induced with stimulating currents, may confirm the precision and safety of the applied currents.

### RELATED APPLICATIONS

The present application is a divisional of U.S. Ser. No. 19/181,619, filed Apr. 17, 2025, the entirety of which is incorporated by reference herein.

### FIELD

This application relates to improvements in the noninvasive modulation of brain electrical activity and function, particularly through cortical surface electrical stimulation. It extends prior invention by providing adaptive synchronization of cortical oscillations (e.g., gamma or spindle-range) for therapeutic purposes (e.g., in neurodegenerative conditions such as Alzheimer's Disease or AD), while providing precise metering of effective electrical dosage and safety mechanisms to prevent adverse excitatory effects such as seizure.

### BACKGROUND

Noninvasive brain stimulation techniques have demonstrated promise in modifying cortical excitability and oscillatory behavior for therapeutic purposes. Transcranial alternating current stimulation (tACS), in particular, enables frequency-specific modulation of brain rhythms. Prior work (U.S. Pat. No. 10,610,121) established the value of estimating and delivering electrical currents in a manner aligned with the orientation of cortical columns. As detailed in that previous invention, quantifying the degree to which the applied current is oriented perpendicular to the person's cortical surface allows more precise determination of the effective stimulation, because the influence of electrical currents is enhanced when aligned with cortical columns (which are perpendicular to the cortical surface) rather than than misaligned. As explained in the inventor's previous invention (U.S. Pat. No. 8,478,011) and publications (e.g., Li, K., Papademetris, X., & Tucker, D. M. 2016. BrainK for Structural Image Processing: Creating Electrical Models of the Human Head, Comput Intell Neurosci, 2016, 1349851) the orientation of the cortical surface can be extracted from the patient's structural MRI, such that the intersection of the applied electrical currents with the cortical surface can be computed precisely (e.g., Hathaway, E., Morgan, K., Carson, M., Shusterman, R., Fernandez-Corazza, M., Luu, P., & Tucker, D. M. 2021. Transcranial Electrical Stimulation targeting limbic cortex increases the duration of human deep sleep. Sleep medicine, 81, 350-357).

Recent advances indicate that synchronizing cortical oscillations at gamma frequencies (30-80 Hz) can enhance cognitive performance and even reduce pathological burden in Alzheimer's Disease. Similarly, entraining sleep spindles (9-16 Hz) during non-REM sleep may support memory consolidation and plasticity. These examples of excitatory modulation carry the risk of inducing pathological synchronization, such as seizure activity, especially in populations predisposed to such events, including those patients with AD.

Thus, there is a need for methods and systems that:
1. Assess specific subsets of cortical columns aligned perpendicularly to the applied electrical current, due to the natural gyral-sulcal folding of the cerebral cortex.
2. Provide frequency-specific synchronization of endogenous brain rhythms.
3. Quantify the degree of entrainment with sufficient precision, such as cortical-surface localized dipolar fields indicating the locally synchronized electrical oscillations.
4. Control the balance of excitatory-inhibitory (E-I) cortical dynamics (as indexed by ongoing cortical oscillations) to maximize benefit and minimize risk.

This invention addresses these needs by extending prior work to allow for the safe, controlled synchronization of orientation-specific cortical oscillations in both waking and sleep states.

### SUMMARY

The invention provides methods and systems for noninvasive synchronization of targeted cortical oscillations by:
Delivering and assessing electrical stimulation aligned with the columnar orientation of the cortex.
Measuring synchronization between external stimulation and intrinsic neural oscillations using EEG, including source localization with high-resolution EEG.
Calculating the quantitative metrics of phase-locking and E-I balance to guide stimulation and assure safety.
Applying adaptive, closed-loop control of stimulation parameters to ensure oscillatory entrainment without triggering hyper-excitatory states.

As illustrative examples, this approach allows therapeutic gamma entrainment in AD patients, or spindle entrainment with slow oscillations during sleep, using the same system with phase and frequency adjustments. In another example, research has shown the ability of slow electrical pulses or waves to reduce the frequency of epileptiform discharges, apparently through inducing long-term depression of cortical activity (Holmes, M. D., Feng. R., Wise, M. V., Ma, C., Ramon, C., Wu, J., . . . . Tucker, D. M. 2019. Safety of slow-pulsed transcranial electrical stimulation in acute spike suppression. Ann Clin Transl Neurol, 6(12), 2579-2585). Critical to the safety and efficacy of these and similar applications is the real-time monitoring of brain activity to ensure synchronization remains within a beneficial regime and does not exceed thresholds that may trigger epileptiform activity.

### DETAILED DESCRIPTION OF THE DRAWINGS

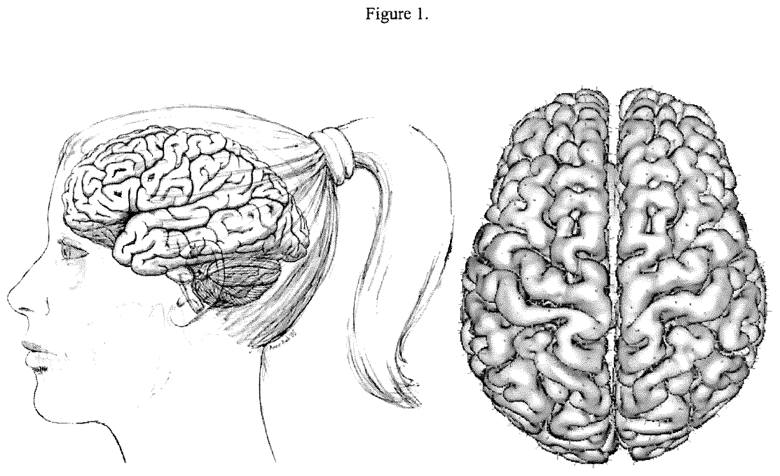

FIG. 1 left illustrates the relationship of the person's cortical surface with the head surface where EEG and TES electrodes are applied. The current paths formed between source and sink TES electrodes are limited, even for a high density electrode array, so that only certain cortical patches will have current perpendicular to the cortical surface, and thus in line with the cortical columns. FIG. 1 right illustrates the tessellation of the cortical surface extracted from the person's MRI at a certain patch density (here 2400 patches), here with a small mark to illustrate the dipole that is positioned at the center of the cortical patch and orthogonal to the net surface orientation of that patch.

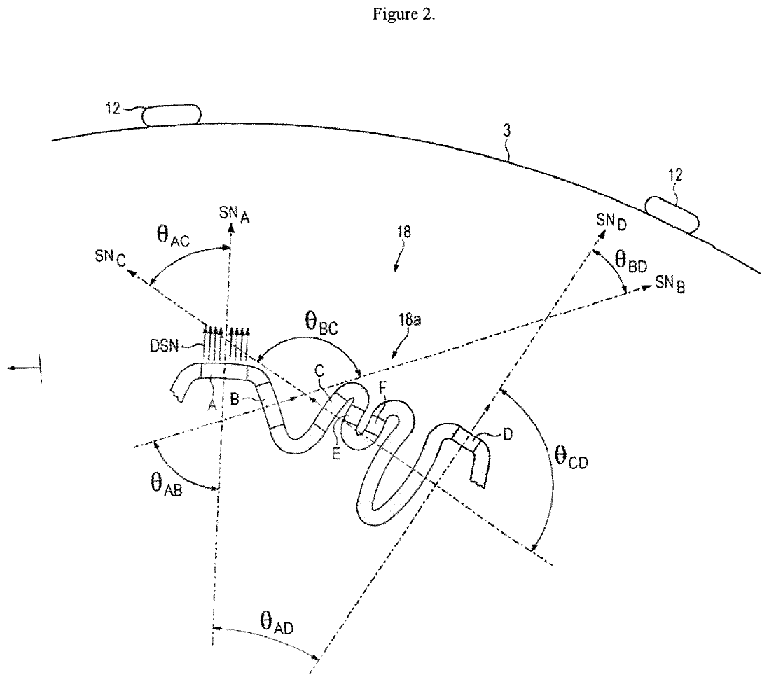

FIG. 2 illustrates the computation between the net orientation of each cortical patch and the induced TES current orientation (drawing from U.S. Pat. No. 10,610,121). U.S. Pat. No. 10,610,121 is incorporated by reference herein in its entirety. The most effective manipulation of cortical function is from current that is parallel to the cortical column (and thus perpendicular to the cortical surface normal (“SN” in FIG. 2) for that patch. Calculating effective dosage of TES is thus done accurately by computing the vector sum of current and surface perpendicular for the cortical patch.

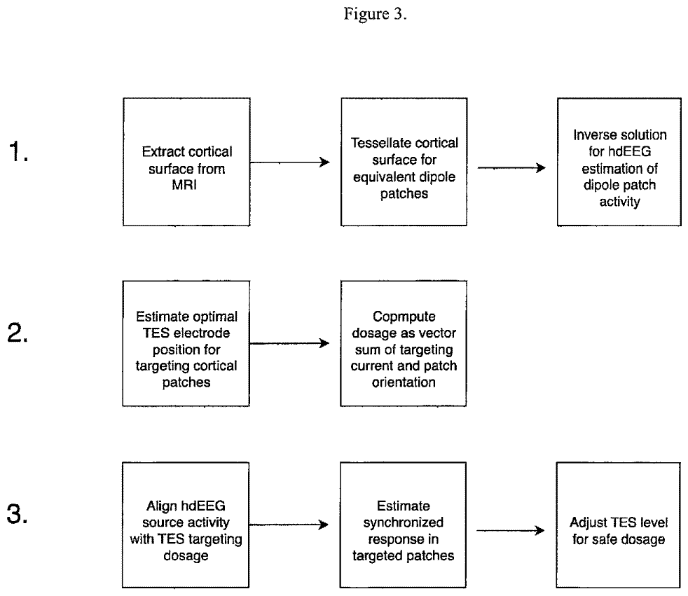

FIG. 3 illustrates the steps in the method. Step 1 is to characterize the patches of the cortex for targeting, such as from the person's MRI (or a probabilistic atlas), through tessellating regular patches on the cortical surface, such as with a graph theory algorithm as described by Li et al (2016) cited above, and developing source localization of the high density EEG (hdEEG) to characterize the activity of each patch as described by Holmes et al (2017) cited above. Step 2 is to estimate the optimal targeting position for the TES electrodes (as described by Hathaway, et al (2021) and to compute the effective dosage of TES with each relevant cortical patch. Step 3 is to adjust the TES dosage based on both the computed effective dosage and the EEG source activity of the targeted area (calibrating the effective dosage for ongoing monitoring).

### DETAILED DESCRIPTION OF A PREFERRED EMBODIMENT

The present invention includes a system composed of:
A multi-channel electrical stimulation device configured for tACS delivery at frequencies influence endogenous brain activity, including frequencies ranging from 0.1 Hz to 1000 Hz.
Image processing software capable of computing/estimating the orientation of cortical columns, either for population norms or for individual participants (such as from structural MRI from a single person or estimated from a population average of cortical orientation, such as from the Montreal Neurological Institute atlas).
A computational model that estimates the effective field strength of the induced TES currents at each cortical location, based on head surface electrode positions, tissue conductivity profiles, and the target orientation of cortical columns.

The stimulation system operates in synchrony with endogenous cortical rhythms by phase-locking its output waveform to measured brain activity. For example:
During waking, gamma-band stimulation is applied to prefrontal or temporoparietal regions with pathological slowing in AD.
During sleep, spindle-range stimulation is applied to frontocentral sites, possibly in phase alignment with slow oscillations, in order to enhance NREM sleep depth and memory consolidation.
In a person with epilepsy, slow pulses may suppress abnormal excitability of target regions to reduce the likelihood of seizure.

To ensure safety, the system calculates a dynamic excitatory-inhibitory (E-I) balance index based on measures of EEG coherence, spectral power, and complexity across relevant frequency bands. If phase-locking or power thresholds exceed pre-defined limits, stimulation is reduced or halted.

Furthermore, individual anatomical models and prior EEG recordings are used to define stimulation maps for each subject, maximizing target precision and avoiding high-risk cortical regions known to propagate seizure activity.

### Adaptive Synchronization of Orientation-Specific Cortical Oscillations for Therapeutic Neuromodulation

### Related Applications

The present application is a divisional of U.S. Ser. No. 19/181,619, filed Apr. 17, 2025, the entirety of which is incorporated by reference herein.

### Field

This application relates to improvements in the noninvasive modulation of brain electrical activity and function, particularly through cortical surface electrical stimulation. It extends prior invention by providing adaptive synchronization of cortical oscillations (e.g., gamma or spindle-range) for therapeutic purposes (e.g., in neurodegenerative conditions such as Alzheimer's Disease or AD), while providing precise metering of effective electrical dosage and safety mechanisms to prevent adverse excitatory effects such as seizure.

### Background

Noninvasive brain stimulation techniques have demonstrated promise in modifying cortical excitability and oscillatory behavior for therapeutic purposes. Transcranial alternating current stimulation (tACS), in particular, enables frequency-specific modulation of brain rhythms. Prior work (U.S. Pat. No. 10,610,121) established the value of estimating and delivering electrical currents in a manner aligned with the orientation of cortical columns. As detailed in that previous invention, quantifying the degree to which the applied current is oriented perpendicular to the person's cortical surface allows more precise determination of the effective stimulation, because the influence of electrical currents is enhanced when aligned with cortical columns (which are perpendicular to the cortical surface) rather than than misaligned. As explained in the inventor's previous invention (U.S. Pat. No. 8,478,011) and publications (e.g., Li, K., Papademetris, X., & Tucker, D. M. 2016. BrainK for Structural Image Processing: Creating Electrical Models of the Human Head. Comput Intell Neurosci, 2016, 1349851) the orientation of the cortical surface can be extracted from the patient's structural MRI, such that the intersection of the applied electrical currents with the cortical surface can be computed precisely (e.g., Hathaway, E., Morgan, K., Carson, M., Shusterman, R., Fernandez-Corazza, M., Luu, P., & Tucker, D. M. 2021. Transcranial Electrical Stimulation targeting limbic cortex increases the duration of human deep sleep. Sleep medicine, 81, 350-357).

Recent advances indicate that synchronizing cortical oscillations at gamma frequencies (30-80 Hz) can enhance cognitive performance and even reduce pathological burden in Alzheimer's Disease. Similarly, entraining sleep spindles (9-16 Hz) during non-REM sleep may support memory consolidation and plasticity. These examples of excitatory modulation carry the risk of inducing pathological synchronization, such as seizure activity, especially in populations predisposed to such events, including those patients with AD.

Thus, there is a need for methods and systems that:
1. Assess specific subsets of cortical columns aligned perpendicularly to the applied electrical current, due to the natural gyral-sulcal folding of the cerebral cortex.
2. Provide frequency-specific synchronization of endogenous brain rhythms.
3. Quantify the degree of entrainment with sufficient precision, such as cortical-surface localized dipolar fields indicating the locally synchronized electrical oscillations.
4. Control the balance of excitatory-inhibitory (E-I) cortical dynamics (as indexed by ongoing cortical oscillations) to maximize benefit and minimize risk. This invention addresses these needs by extending prior work to allow for the safe, controlled synchronization of orientation-specific cortical oscillations in both waking and sleep states.

### Summary

The invention provides methods and systems for noninvasive synchronization of targeted cortical oscillations by:
Delivering and assessing electrical stimulation aligned with the columnar orientation of the cortex.
Measuring synchronization between external stimulation and intrinsic neural oscillations using EEG, including source localization with high-resolution EEG.
Calculating the quantitative metrics of phase-locking and E-I balance to guide stimulation and assure safety.
Applying adaptive, closed-loop control of stimulation parameters to ensure oscillatory entrainment without triggering hyper-excitatory states.

As illustrative examples, this approach allows therapeutic gamma entrainment in AD patients, or spindle entrainment with slow oscillations during sleep, using the same system with phase and frequency adjustments. In another example, research has shown the ability of slow electrical pulses or waves to reduce the frequency of epileptiform discharges, apparently through inducing long-term depression of cortical activity (Holmes, M. D., Feng, R., Wise, M. V., Ma, C., Ramon, C., Wu, J., . . . . Tucker, D. M. 2019. Safety of slow-pulsed transcranial electrical stimulation in acute spike suppression. Ann Clin Transl Neurol, 6(12), 2579-2585). Critical to the safety and efficacy of these and similar applications is the real-time monitoring of brain activity to ensure synchronization remains within a beneficial regime and does not exceed thresholds that may trigger epileptiform activity.

### Detailed Description of the Drawings

FIG. 1 left illustrates the relationship of the person's cortical surface with the head surface where EEG and TES electrodes are applied. The current paths formed between source and sink TES electrodes are limited, even for a high density electrode array, so that only certain cortical patches will have current perpendicular to the cortical surface, and thus in line with the cortical columns. FIG. 1 right illustrates the tessellation of the cortical surface extracted from the person's MRI at a certain patch density (here 2400 patches), here with a small mark to illustrate the dipole that is positioned at the center of the cortical patch and orthogonal to the net surface orientation of that patch.

FIG. 2 illustrates the computation between the net orientation of each cortical patch and the induced TES current orientation (drawing from U.S. Pat. No. 10,610,121). U.S. Pat. No. 10,610,121 is incorporated by reference herein in its entirety. The most effective manipulation of cortical function is from current that is parallel to the cortical column (and thus perpendicular to the cortical surface normal (“SN” in FIG. 2) for that patch. Calculating effective dosage of TES is thus done accurately by computing the vector sum of current and surface perpendicular for the cortical patch.

FIG. 3 illustrates the steps in the method. Step 1 is to characterize the patches of the cortex for targeting, such as from the person's MRI (or a probabilistic atlas), through tessellating regular patches on the cortical surface, such as with a graph theory algorithm as described by Li et al (2016) cited above, and developing source localization of the high density EEG (hdEEG) to characterize the activity of each patch as described by Holmes et al (2017) cited above. Step 2 is to estimate the optimal targeting position for the TES electrodes (as described by Hathaway, et al (2021) and to compute the effective dosage of TES with each relevant cortical patch. Step 3 is to adjust the TES dosage based on both the computed effective dosage and the EEG source activity of the targeted area (calibrating the effective dosage for ongoing monitoring).

### Detailed Description of a Preferred Embodiment

The present invention includes a system composed of:
A multi-channel electrical stimulation device configured for tACS delivery at frequencies influence endogenous brain activity, including frequencies ranging from 0.1 Hz to 1000 Hz.
Image processing software capable of computing/estimating the orientation of cortical columns, either for population norms or for individual participants (such as from structural MRI from a single person or estimated from a population average of cortical orientation, such as from the Montreal Neurological Institute atlas).
A computational model that estimates the effective field strength of the induced TES currents at each cortical location, based on head surface electrode positions, tissue conductivity profiles, and the target orientation of cortical columns.

The stimulation system operates in synchrony with endogenous cortical rhythms by phase-locking its output waveform to measured brain activity. For example:
During waking, gamma-band stimulation is applied to prefrontal or temporoparietal regions with pathological slowing in AD.
During sleep, spindle-range stimulation is applied to frontocentral sites, possibly in phase alignment with slow oscillations, in order to enhance NREM sleep depth and memory consolidation.
In a person with epilepsy, slow pulses may suppress abnormal excitability of target regions to reduce the likelihood of seizure.

To ensure safety, the system calculates a dynamic excitatory-inhibitory (E-I) balance index based on measures of EEG coherence, spectral power, and complexity across relevant frequency bands. If phase-locking or power thresholds exceed pre-defined limits, stimulation is reduced or halted.

Furthermore, individual anatomical models and prior EEG recordings are used to define stimulation maps for each subject, maximizing target precision and avoiding high-risk cortical regions known to propagate seizure activity.

1. A method for safely applying excitatory cortical stimulation in Alzheimer's Disease patients comprising:
determining an individualized excitatory-inhibitory balance threshold for each patient,
measuring the deviation from this threshold during stimulation,
and automatically adjusting stimulation parameters to avoid exceeding the seizure-critical limit.

2. A non-transitory computer-readable medium storing instructions that, when executed, control the modulation of cortical electrical stimulation based on individualized modeling of cortical column orientation, E-I balance, oscillatory phase-locking, and seizure risk thresholds.
